(19) United States
(12) Patent Application Publication
(10) Pub. No.: US 2011/0008894 A1
(43) Pub. Date: Jan. 13, 2011

(54) LYOPHILIZED PLASMID/DNA TRANSFECTION REAGENT CARRIER COMPLEX

(75) Inventors: Emmanuel RAVET, Airoux (FR); Fabienne VERNEJOUL, Toulouse (FR); Jean-Paul REYNES, Escalquens (FR); Daniel DROCOURT, Saint Orens (FR); Gérard TIRABY, Salade-Ponsan (FR); Eric PEROUZEL, Toulouse (FR)

Correspondence Address: YOUNG & THOMPSON, 209 Madison Street, Suite 500, Alexandria, VA, 22314, US

(73) Assignee: CAYLA, Toulouse (FR)

(21) Appl. No.: 12/830,614

(22) Filed: Jul. 6, 2010

Related U.S. Application Data
(60) Provisional application No. 61/223,530, filed on Jul. 7, 2009.

(51) Int. Cl. C12N 15/63

(52) U.S. Cl. 435/455; 435/320.1

(57) ABSTRACT

Disclosed is a novel formulation for the production of a lyophilized plasmid/DNA transfection reagent complex capable of serving as a carrier for additional free plasmids. Upon rehydration, this plasmid/DNA transfection reagent carrier can be used to introduce simultaneously the complexed plasmid and the additional free plasmids into animal cells. This novel formulation can be useful for viral particle production, gene transfer experiments like gene silencing experiments, reporter gene, or integration/selection experiments.

### FIELD OF INVENTION

The present invention relates to transfection reagents and compositions of transfection reagents to deliver nucleic acids into cells.

### BACKGROUND

Transfection refers to the introduction of DNA into a recipient eukaryote cell. Usually accomplished using DNA complexed with cationic lipids, also referred to as liposomes, or cationic polymers, although a variety of other methods can be used, such as electroporation or calcium ions. Cationic lipids and cationic polymers are widely under investigation as non-viral transfectants to introduce DNA into a target cell (Behr et al., 1994; Cotton et al., 1993). For transfection purposes, cationic lipids can be mixed with a non-cationic lipid, usually a neutral lipid, to increase transfection efficiency or stability. Typically, the helper lipids are cholesterol or dioleoylphosphatidylethanolamine (DOPE), 1,2-diphytanoyl-sn-glycero-3-phosphoethanolamine (DiPPE) to improve transfection efficiencies and/or colloidal stability in vitro (Felgner et al., 1994). The most widely studied non-liposomal cationic polymeric vector is linear polyethylenimine (PEI; Intra and al, 2008).

Cationic lipids and cationic polymers complexed with nucleic acids, are called lipoplexes and polyplexes, respectively (Felgner et al., 1997). The complexes are formed through the interaction of the cationic charges of lipids or polymers with the negative charge on DNA. These complexes render nucleic acids resistant to DNAses and condense the DNA into fusogenic nanoparticles. These properties make cationic polymers or lipids interesting delivery vehicles as DNA plasmid alone is not able to enter into cell. By carefully choosing the cationic lipid or cationic polymer, it is therefore possible to transfect cells with a gene of interest.

For any chosen transfection reagent, multiple formulation parameters have been reported to significantly affect the transfection efficiency of lipoplexes or polyplexes. The formulation parameters with greatest influence on transfection efficiencies are lipid/polymer to DNA ratio, charge ratio and particle size (Birchall et al., 1999).

Transfection reagents are usually used in excess of the cationic mixture to induce complete complexation of the DNA into a positively charged particle (Masotti et al., 2008). The resulting aggregate of complexed DNA is then able to interact with the negatively charged cell surface proteogylcans (Ewert et al. 2005; Kopatz et al., 2004) and can therefore be internalized by the cell for expression of the DNA. However, as the excess of lipid or polymer needs to be limited to reduce cellular toxicity, an optimum ratio is determined for each transfection reagent.

Moreover, it was found that transfection efficiency is significantly influenced by lipoplex or polyplex particle size (Masotti et al., 2008). In general large particles showed higher transfection efficiency than smaller ones. Bigger size is usually obtained by the addition of salt during the formulation process. Most commercial DNA transfection reagents (Lipofectamine™ 2000, Invitrogen, Carlsbad, Calif., USA; FuGENE® HD, Roche Applied Sciences, Indianapolis, Ind., USA; Effectene™, QIAGEN, Germantown, Md., USA; jetPEI™, PolyPlus, Illkirch, France) are therefore mixed prior to use with DNA in cellular medium, like OptiMeM or PBS, containing a great quantity of salt. Bigger particles are more prone to sediment on top of adherent cells and therefore increase contact between cells and DNA. They are also thought to be less tightly packed (loosely grouped) when inside the cell where they need to release the DNA (Turek et al., 2000). One drawback of using a large particle is the resulting colloidal instability that is incompatible with use in a clinical setting. With such an unstable formulation, one can only prepare the lipoplexes or polyplexes prior to use. This has been a major limitation for clinical applications, where mixing prior to use is not technically safe, feasible (therapeutically relevant concentration cannot be attained) or can generate significant irreproducibility.

This high instability of a liquid formulation has stimulated considerable interest in developing lyophilized formulation that could be stored at room temperature and simply rehydrated by the clinician prior to use (Allison and Anchordoquy, 2000; Anchordoquy et al., 1997; Cherng et al., 1999; Talsma et al., 1997). An added benefit of such lyophilized formulations would be the ability to produce large standardized batches required for commercial use or any GMP production prior to use in a clinical setting. Several studies have investigated the parameters allowing for both appropriate transfection efficiency and lyophilization. Maintenance of particle size after rehydration appears to be a key parameter that can be achieved by using lyoprotectant, such as sucrose (Li et al., 2000). Clearly, in these experiments, the researcher is required to choose their plasmid of interest prior to lyophilization. For example, U.S. Pat. Nos. 6,726,926 and 7,276,359 disclose lyophilized and frozen compositions of liposomes and polynucleotides.

Some transfection experiments may require multiple plasmids with one or more reference plasmids remaining constant. In the first instance, this reference plasmid codes for a reporter gene. Researchers working with cells of unknown or variable transfection efficiencies use a plasmid coding for a reporter gene, such as green fluorescent protein (GFP), secreted alkaline phosphatase (SEAP) or luciferase (luc), as an indicator of transfection efficiency, in combination with another plasmid coding for the gene of interest. This also constitutes an internal control of particular interest in silencing experiments when using an RNAi coding variable plasmid of unknown cellular effect or cytotoxicity. In other settings, such as integration studies, the reference plasmid codes for the integrase or transposase, and the variable plasmid codes for the gene of interest to be integrated flanked by the appropriate recognition sequences. Another example is an experimental setting where the reference plasmid codes for proteins involved in viral particle production like lentivirus production. In such an experiment, the lentiviral accessory genes are placed on two or more plasmids. The variable plasmid is then coding for the transgene of interest, which is to be inserted into the viral derived vector genome.

One of the most popular viral-based gene transfer methods involves lentiviral vectors. These vectors have attracted the attention of researchers because they can transduce with a high efficiency a wide range of cell types, non-discriminately transducing both dividing and non-dividing cells. As opposed to other popular vector delivery systems, lentivirus stably and rapidly integrates the transgene into the host genome allowing for long-term studies in vivo, and stable expression of transgenes and with appropriate genes the generation of immortalized cell lines. Current lentiviral vector systems can accommodate upwards of ten kilobases of foreign DNA (Zufferey et al., 1997), although promoter and enhancer elements reduce the practical size of gene open-reading frames to up to seven kilobases, which is sufficient to accommodate most genes commonly studied in the mammalian genome. Cell tropism is expanded via pseudotyping usually with vesicular stomatitis virus G (VSV-G) envelope glycoproteins (Burns et al., 1993; Russell and Miller, 1996).

While lentiviral vectors provide many advantages, one of their prime advantages, the ability to stably integrate into a host cell's chromosomes, can also be a major safety concern. This ability to integrate into a chromosome can cause insertional mutagenesis (Verma and Somia, 1997). One method of dealing with this problem has been to fuse a specific DNA binding domain to the integrase (IN) polypeptide to direct integration into specific DNA sequences (Bushman, 1995; Bushman and Miller, 1997; Katz et al., 1996). Moreover, there are many instances where one does not want to have a gene stably integrated, but only transiently expressed for a limited time period. For example in cancer therapy, such an approach is useful with “suicide therapy” where the gene product is designed to negatively impact the integrity of the host cell. One type of expression where a gene is not integrated into a chromosome is episomal replication. It would be desirable to have an episomal replicating vector. Lentiviruses and lentiviral vectors can be rendered integration defective by mutations in the integrase coding sequence or altering the integration recognition sequences (att sites) in the viral LTR (Nightingale et al., 2006). Recent in vitro studies have shown that integration-deficient lentiviral vectors can mediate transduction of genes whose expression quickly fade away upon incubation time of dividing as well as non dividing recipient cells (Lu et al., 2004; Saenz et al., 2004; Vargas et al., 2004; Yanez-Munoz et al., 2006). Furthermore, prior research has demonstrated that a mutation in the integrase gene of a lentiviral vector had no detectable effect on the various steps of the infection pathway, including particle budding, entry into the target cell, reverse transduction, and nuclear transport (Naldini et al., 1996).

Lentivectors are generated using a ‘split-component’ production system, the overall objective being to make each component less and less complete in function, to the point where infectious viral particles can only be produced in the packaging cell and not from the final vector preparation. Typically, producer cell lines are transfected with (i) the vector plasmid, coding for the transgene, lentiviral LTRs for host cell integration and perhaps the Rev-responsive element (RRE) for most efficient vector production; (ii) a plasmid encoding the gag and poi viral structural genes, in order to supply reverse transcriptase and integration functions for the therapeutic vector particles; and (iii) plasmids encoding envelope proteins for the therapeutic viral particles and perhaps Rev protein. Three generations of lentiviral packaging systems have been successively developed. The first generation encompasses all HIV-1 genes except the envelope. In the second generation system all the viral auxiliary genes have been deleted. Third generation lentiviral vectors only uses a fractional set of HIV genes: only gag, pol, with rev provided on a separate plasmid (Dull et al., 1998). For optimal safety consideration, the third generation of lentivirus affords the highest level of protection, whereby minimal genetic elements are split among three or four plasmids that must be expressing simultaneously in individual cells for successful viral production. Any potential for recombination between HIV-derived constructs is greatly reduced.

Evolution of the lentiviral vector production has not been confided to modifications in the packaging plasmids, the lentiviral expression vector has also been altered. This vector expresses the full-length vector RNA, containing all the cis-acting elements, required for efficient packaging, reverse transcription, nuclear import and integration) and the transgene expression cassette (internal promoter and transgene sequence). The original model for subsequent lentiviral vectors was an HIV-1 Tat-dependent vector expressing full-length vector mRNA from the 5′ LTR (long terminal repeat) and terminating in the 3′ LTR. Replacement of the U3 in the 5′ LTR with a potent heterologous promoter rendered the vector Tat-independent, and thus, enabled vector production with a third generation packaging system. Increased vector titers were obtained by substituting a cellular polyadenylation signal for the 3′U5. A critical improvement in vector safety was the deletion of the enhancer/promoter sequences in the U3 region of the 3′LTR, which defines the vectors as self-inactivating (SIN) vectors. The deletion in the 3′ LTR allows SIN lentivectors to productively infect and integrate into target cell populations, but generation of proviral transcripts is blocked. Improvements in transgene expression and transduction efficiency of lentiviral vectors were accomplished through the incorporation of the woodchuck hepatitis virus post-transcriptional regulatory element (WPRE), and the central purine polypurine tract (cPPT). When placed in the sense orientation in the 3′ untranslated region of a transgene, the WPRE sequence increases overall transgene expression by more than five-fold (Zufferey et al., 1999). The efficiency of HIV-1 was increased through the addition of cPPT and central termination sequence (CTS) (Zennou et al., 2000).

After transfection and subsequent expression of the proteins encoded by the transiently transfected plasmids, the cell medium supernatant contains active lentiviral particles immediately usable to transduce other cells of interest. Problems with reproducibility, low titer and toxicity in purified preparations plague common usage of lentiviral vectors. Traditional methods for production of lentiviral titers utilize calcium phosphate precipitation to transfect the required plasmids into a packaging cell line, such as 293T human embryonic kidney cells (ATCC cat. no. CRL-11268) (Kutcher et al., 2009). In our experience, this standard approach leads to high variability in transfection efficiencies and corresponding titers, and the concentrated virus can be highly toxic to cells in downstream experiments. The lack of reproducibility may be in part due to the use of freshly made calcium chloride. More recently, other methods have been developed using more conventional transfection reagents, such as PEI or jetPEI, and commercially available transfection reagents of proprietary composition, such as Lipofectamine™ 2000, and FuGENE® HD (Biotechniques Protocol Guide, 2009).

In commercial kits, the mix of plasmids required for packaging of lentiviral vectors is available in liquid form, with Lenti-X HT packaging mix from Clontech or in lyophilized form with ViraPower™ Packaging Mix from Invitrogen. These plasmids then need to be, combined with the expression vector together with a transfection reagent (Lentiphos™ HT or Lipofectamine 2000™) in a media of choice prior to use. This formulation step is by nature highly variable as multiple parameters interplay with each other like the aging of the transfection reagent or the media, incubation conditions (temperature, time), purity and concentration of the plasmids, volume of the preparation, and accurate determination of plasmid ratio. These formulation parameters combined with other important parameters related to the state of the cells used for production render the lentiviral production process highly variable. It is not unusual in the lab to observe multiple-log variations in titers. It would, therefore, be of great benefit for the user if the biotechnology industry could provide for a technical solution reducing the fluctuation of the formulation parameters and allowing for a more reproducible production of high-titer production.

As mentioned previously, one could expect that lyophilization of the transfection reagent with the plasmids would allow for this highly desirable standardization. Such a multiple plasmid formulation has never been described but could be envisaged by one skilled in the art of lipoplexes/polyplexes formulation. As researchers are working with different genes of interest related to their own field of research, a lyophilizate for each possible gene combination would be need to be produced; the expression plasmids would need to be incorporated with the transfection reagent prior to lyophilization. This lack of flexibility would render the use of such a lyophilizate very improbable as it would not be cost or time effective to produce a customized lyophilizate for each user.

It would, therefore, be of great interest to design a lyophilized formulation containing a mix of the packaging plasmid and the envelope plasmid together with a transfection reagent that could act as a carrier for an expression vector chosen by the researcher from the many different types available. Such a technical solution would reduce the fluctuation of the transfection formulation parameters to a minimum, as the researcher would only have to add their expression plasmid into a fresh suspension of the carrier. The benefits of this lyophilized carrier will be of even greater importance in settings where reproducibility is essential, such as the good manufacturing production (GMP) for clinical use. Thus there is a continuing need in the art for improved formulations and methods for delivery of genes to animals and humans.

The novel element of the disclosed patent is the ability to add further plasmid DNA to a lyophilizate of lipoplexes or polyplexes. This further DNA is an integral part of the experimental studies. Ideally, this formulation would be in the form of a lyophilized powder, to which a researcher would only need to add the expression plasmid coding for the gene of interest. Such a formulation will have numerous advantages, such as ease-of-storage, stability, large scale production, ease-of-use, reproducibility of a standardized formulation and good manufacturing practice (GMP) compliance. The present invention overcomes difficulties of reproducibility by allowing use of a readymade solution that can be produced industrially. In the case of viral production, the invention provides other advantages, such as increased viral titer and reduced the necessary experimental time to produce virions.

### SUMMARY OF INVENTION

The present invention provides a biologically active transfection formulation for the preparation of highly lyophilization-stress resistant, hydratable lipoplex/polyplex lyophilizates and methods for their reconstitution. According to the invention, biologically active compositions refer to a transfection formulation comprising a DNA transfection reagent and plasmids, wherein said product serves as a “carrier” for one or more additional free plasmids, and is used to introduce complexed plasmids and additional free plasmids simultaneously into animal cells.

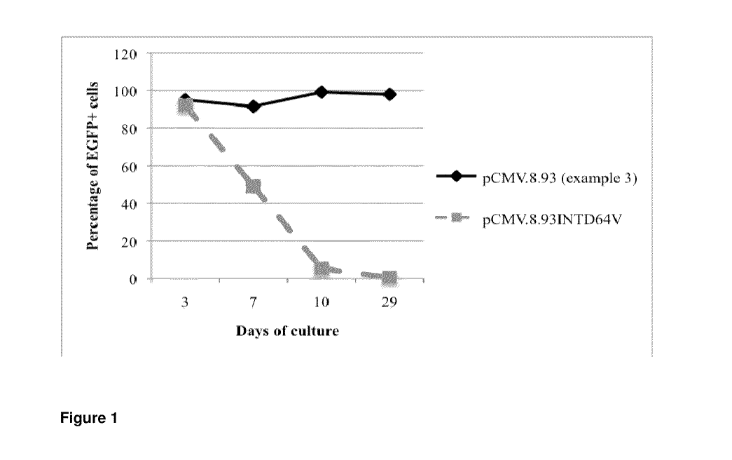

### BRIEF DESCRIPTION OF FIG. 1

FIG. 1. shows expression of EGFP in HT1080 transduced cells at different times of culture. Reduction of EGFP expression over time following cell division is consistent with production of an non-integrative vector.

### DETAILED DESCRIPTION OF INVENTION

The present invention relates to a method for transfecting an animal cell, comprising the steps of:

a) rehydrating a lyophilized composition comprising a lyoprotectant and one or more plasmids complexed with a DNA transfection reagent;

b) adding to the rehydrated composition obtained in step a) one or more free plasmids; and

c) transfecting an animal cell with the mixture obtained in step b).

The present invention provides a formulation of multiple plasmids complexed with a DNA transfection reagent in a single lyophilized preparation. Upon rehydration, this lyophilizate is an efficient DNA transporter capable of transfecting animal cells. Furthermore, this lyophilizate also serves as a “carrier” for one or more additional free plasmids. Upon rehydration with additional free plasmids, this carrier can be used to introduce the complexed plasmids, contained in the lyophilizate, and the additional free plasmids simultaneously into animal cells, 293T human embryonic kidney cells (ATCC cat. no. CRL-11268) are used in the examples presented. Note that any similarly immortalized cell line can be used for this type of work; HeLa, COS and Chinese Hamster Ovary (CHO) cell are common alternatives.

The current invention includes the use of a transfection reagent, either a cationic lipid with or without a neutral helper lipid or a cationic polymer.

An example of cationic lipid-based formulation is LyoVec™. LyoVec™ is a cationic lipid-based transfection reagent commercially available from InvivoGen. The major constituent of LyoVec™ is the phosphonolipid di-tetradecylphosphoryl-N,N,N-trimethylmethanaminium chloride (DTCPTA) which is combined with the neutral lipid 1,2-diphytanoyl-sn-glycero-3-phosphoethanolamine (DiPPE).

An example of a cationic polymer is polyethyleneimine (PEI), in particular jetPEI™, commercially available from PolyPlus (Illkirch, France).

In addition, the complex requires the presence of a lyoprotectant during the lyophilization process, in order to retain the transfection efficiency of the complex upon rehydration.

Examples of lyoprotectant are the carbohydrate cryoprotectants such as sucrose, glucose, lactose, trehalose, arabinose, pentose, ribose, xylose, galactose, hexose, idose, monnose, talose, heptose, fructose, gluconic acid, sorbitol, mannitol, methyl [alpha]-glucopyranoside, maltose, isoascorbic acid, ascorbic acid, lactone, sorbose, glucaric acid, erythrose, threose, arabinose, allose, altrose, gulose, erythrulose, ribulose, xylulose, psicose, tagatose, glucuronic acid, gluconic acid, glucaric acid, galacturonic acid, mannuronic acid, glucosamine, galactosamine, neuraminic acid, arabinans, fructans, fucans, galactans, galacturonans, glucans, mannans, xylans, levan, fucoidan, carrageenan, galactocarolose, pectins, pectic acids, amylose, pullulan, glycogen, amylopectin, cellulose, dextran, pustulan, chitin, agarose, keratin, chondroitin, dermatan, hyaluronic acid, alginic acid, xanthin gum, starch and combinations thereof.

The preferred lyoprotectant is saccharose, glucose, lactose, trehalose, or combinations thereof.

Typically, the concentration prior to lyophilization of the lyoprotectant varies from 0.5 to 10% (w/v), where the preferred concentration is about 2.5% (w/v).

The transfection reagent can also further comprise a salt, such as sodium chloride. Typically, the concentration prior to lyophilization of salt lies within the range 0.2-1% (w/v), where the preferred concentration is about 0.45% (w/v).

It falls within the ability of one skilled in the art to find the adequate conditions for the complexation of the supplementary DNA. Typically ratio of transfection reagent to DNA would have to be adjusted to meet the formulation issue such as the inability to complex supplementary DNA, precipitation, inability to re-suspend the lyophilizate. Typically, in the case of LyoVec, a very atypical anionic ratio should be used in order to obtain the desired properties. The resulting particles had to be used in greater quantities than usual to transfect efficiently (3 times more DNA). In the case of linear PEI (jet PEI™), the standard in vitro charge ratio was kept, the salt could be completely removed, and glucose was used. The typical steps would involve evaluation of multiple DNA ratios in variable salt and lyoprotectant conditions. One would have also to adjust quantity of DNA added to the cells to generate efficient transfection with acceptable toxicity.

One application of this invention is the production of viral vectors and in particular lentiviral vectors. The present invention encompasses both integrating and non-integrating lentiviral vectors. In the present invention, the formulation can be used for the production of replication-defective HIV particles, through the use of a producer cell line. The producer cell line is of HEK 293T cell type, such as 293T cells (ATCC, cat no. CRL-11268). All the genes necessary for lentiviral vector production are contained in the packaging plasmid(s). Preferably, there is at least one vector containing nucleic acid sequences encoding i) the lentiviral pol proteins necessary for reverse transcription and integration, ii) lentiviral gag protein necessary for forming a viral capsid, iii) rev protein which binds to the RRE (rev responsive element) and facilitates export of viral RNA in the viral capsid, iv) tat gene which is a transcriptional transactivator which binds to the TAR sequence in the LTR. Preferably, the lentiviral vector is a form of self-inactivating (SIN) vector as a result of a deletion in the 3′ long terminal region (LTR).

An innovative process characterized by its simplicity and efficacy has been developed for the production of lentiviral particles on a small scale from the adherent cells of the 293T cell line. This process can be further adapted for the production of lentiviral particles on larger scales from the 293T Cells grown in suspension in a serum free defined medium. This process is suitable for complete adaption to a manufacturing process of lentiviral particles under the good manufacturing guidelines (GMP). Since lyophilization is a commonly used process to render biological reagents readily transportable and storage stable, this finding has significant ramifications.

In another embodiment of the present invention a kit is provided, which comprises:

a) a lyophilized composition comprising a lyoprotectant and one or more plasmids complexed with a DNA transfection reagent, and

b) an expression vector comprising a cloning site which enables the introduction of a nucleotide of interest.

Typically a viral particle production kit according to the invention comprises:

a) a lyophilized composition comprising a lyoprotectant and one or more plasmids coding for lentiviral accessory and pseudotyping genes, such as gag, tat, pol, rev and VSV-G, complexed with a DNA transfection reagent; and.

b) an expression vector comprising a cloning site which enables the introduction of a nucleotide sequence coding for a gene of interest or an RNA of desirable biological activity like an RNAi or miRNA.

Typically a selection/integration experiment kit according to the invention comprises:

a) a lyophilized composition comprising a lyoprotectant and a plasmid complexed with a DNA transfection reagent, wherein said plasmid codes for a protein selected from the group consisting of a recombinase, a transposase a transcription factor, a DNA repair protein, a repressor, a transactivating factor, a zinc-finger protein, a leucine-zipper protein, a cell cycle protein, a meganuclease, a DNA polymerase, and a DNA ligase.

b) an expression vector containing a multiple cloning site or a nucleotide of interest flanked by a nucleotide sequence that is recognized by said protein.

Such a kit would offer a ready to use solution for experiments requiring the use of a protein together with a nucleic acid sequence. The expected benefit for the user would be a standardization of the transfection conditions increasing therefore reproducibility of results. For example, when performing an integration experiment, ratio of the expression vector to transposase needs to be optimized to avoid overproduction inhibition. This parameter can be standardized by using such a kit and therefore would avoid cumbersome optimization by the end-user.

Typically a transfection kit according to the invention comprises:

a) a lyophilized composition comprising a lyoprotectant, and a plasmid coding for a reporter gene, such as green fluorescent protein (GFP) or any gene which could serve to normalize the transfection experiments results, complexed with a DNA transfection reagent; and.

b) an expression vector comprising a cloning site which enables the introduction of a nucleotide sequence of interest (e.g., a gene of interest or a nucleotide sequence encoding a RNA of desirable biological activity like an RNAi or miRNA).

The main advantages of the present invention are that it is simple-to-use, reproducible, GMP compliant, offering maximum transfection efficiency, with minimal cytotoxicity and increased stability of the pDNA/transfection reagent complexes. The lyophilizate can be resuspended due to the optimization of the concentration of salts, sugars and DNA:lipid ratio. One of the major applications of this invention is as a “carrier” comprising a lyophilized mixture containing all the elements necessary for transfection with the exception of the plasmid coding for the transgene of interest. The advantage of this approach is that researchers have a ready-to-use system and operator variability is reduced, therefore, increasing reproducibility. For example, with viral particle production, a lyophilizate containing transfection reagent and the plasmids coding for the accessory viral genes could be produced, to which a plasmid coding for the transgene is simply added prior to transfection.

The following examples illustrate some embodiments of the present invention in detail. These examples are merely illustrative of the present invention and should not be considered as limiting the scope of the invention in any way.

### Example 1

### Preparation of a Lyophilized Powder of Multiple DNA Plasmids and Transfection Reagent

The aim of this example is to illustrate that a DNA transfection reagent and multiple plasmids can be lyophilized, and more importantly, that upon rehydration that this lyophilizate provides an efficient means to transfect animal cells. It is known from prior art that one plasmid complexed with a transfection reagent can be lyophilized and upon rehydration used to transfect animal cells. In this example, we demonstrated that multiple plasmids in combination with a DNA transfection reagent can be lyophilized, and retain their ability to efficiently transfect animal cells upon rehydration.

### Lyophilization of Transfection Reagent and 3 Plasmids

Plasmid DNA was purified using QIAGEN Plasmid Maxi kit (cat. no. 12162). This example consists of a lyophilized powder containing DTCPTA/DiPPE (components of LyoVec™ transfection reagent; InvivoGen) and three DNA plasmids, each coding for a different reporter gene. The first plasmid is pDRIVE5-GFP-1 (3.604 kb; InvivoGen, cat. no. pdv5-gfp-1) coding for the green fluorescent protein. The second plasmid is pORF-hSEAP (4.691 kb InvivoGen, cat. no pORF-hSEAP) coding for a secreted human alkaline phosphatase. The third plasmid is pCMV-GLuc (5.76 kb; New England Biolabs, cat. no N8081S) coding for the secreted Gaussia luciferase.

The lyophilizate was prepared as follows; 15 μg pDRIVE5-GFP-1, 15 μg pORF-hSEAP, and 15 μg pCMV-GLuc was added to a 1 ml solution containing DTCPTA/DiPPE (125 μg/ml) in 0.45% (w/v) NaCl and 2.5% (w/v) saccharose. 500 μl of the mixture was aliquoted into two vials and placed at −80° C. for 24 hours prior to lyophilization. Lyophilization was carried out as follows, 20 hours at −30° C., 6 hours at −20° C., 8 hours at −10° C., and 6 hours at 35° C. The ratio of DNA:lipid is of 1:2.7 (w/w) and has a negative charge. The lyophilizate was rehydrated with 1 ml of sterile water (final conc 22.5 μg/ml total DNA).

### Freshly Prepared Transfection Mixture

LyoVec™ transfection reagent (LyoVec™, InvivoGen, cat no. lyec-1; DTCPTA/DiPPE 60 μg/ml in 1.8% (w/v) NaCl and 10% (w/v) saccharose) was rehydrated with 2 ml of sterile water. A lipoplex was freshly prepared as follows: 3.33 μg pDRIVE5-GFP-1, 3.33 μg pORF-hSEAP, and 3.33 μg pCMV-GLuc was added to 1 ml of rehydrated LyoVec™ transfection reagent (60 μg/ml) and left to incubate for 15 minutes at room temperature. The ratio of DNA:lipid is 1:6 (w/w) and is positively charged. The final concentration of this mixture is 10 μg/ml total DNA.

### Transfection of 293T Cells

Transfection of 293T Cells (ATCC, cat no. CRL-11268) was carried out as follows: 300,000 cells/well were seeded in a 12-well plate, to which 100 μl of complex (2.25 μg total DNA for the lyophilized transfection reagent and plasmids) was added directly into the culture medium drop wise and mixed by rocking the culture plate back and forth. For the freshly prepared transfection mixture, 1 μg of total DNA was added per well as per instructed by manufacturer for optimal transfection efficiency. 24 hours after transfection, the culture media was replaced with fresh DMEM (Invitrogen, cat. no. 10313039) containing 10% heat inactivated fetal bovine serum (FBS; Invitrogen, cat. no. 10438018). Heat inactivated FBS was used because alkaline phosphate present in serum could interfere with the quantification of SEAP. Unlike other transfection reagents, LyoVec™ is not toxic to cells. The cells were further incubated at 37° C. for a total of 48 hours to allow expression of the GFP, SEAP, and Gaussia luciferase transgenes.

### Evaluation of Reporter Gene Expression

GFP expression was first evaluated by simple visualization under a fluorescent microscope and then assessed using a microplate reader (FLUOStar OPTIMA, BMG laboratories) with filter excitation at 485 nm and emission at 528 nm. Detection and quantification of SEAP in cell culture supernatant was carried out using QUANTI-Blue™ (InvivoGen, cat. no. rep-qb1) according to manufacturer instructions. Briefly, 20 μl of supernatant from the transfected cells was added to 180 μl of QUANTI-Blue™ and incubated at 37° C. for 1-3 hours. The SEAP activity in cells was measured using microplate reader (FLUOStar OPTIMA, BMG laboratories), filter with excitation at 620 nm and emission at 655 nm. Gaussia luciferase assay was performed with 8 μl of stabilizer and 20 μl of supernatant and 50 μl of Gaussia luciferase assay reagent (New England Biolabs, cat. no. E3300S) and results were expressed using the above plate reader as Relative Light Unit (RLU).

TABLE 1
showing % transfection efficiencies GFP, SEAP
and luciferase for lyophilized
product compared to a freshly prepared product. Mean ±
SD from three independent
transfectionexperiments.

| | Lyophilized | Freshly prepared |
|---|---|---|
| | product | product |
| GFP (fluorescence Abs, 485 | 4,500 ± 600 | 5,000 ± 400 |
| nm) | | |
| SEAP(O.D. 650 nm) | 1.2 ± 0.2 | 1 ± 0.3 |
| Luciferase (RLU) | 300 000 ± 400 | 250 000 ± 500 |

Experiments have been repeated with 2 months and six months old lyophilized samples hold at 4° C. with essentially identical results as reported in Table 1.

### Conclusion

The results indicate that the transfection efficiencies were comparable for the lyophilized product and the freshly prepared product, based on the fluorescence intensities for the GFP protein, optical density readings for SEAP, and luminescence measurement for Gaussia luciferase. It is therefore possible to lyophilize a mix of several DNA plasmids together with a transfection reagent in appropriate condition and successfully transfect 293T Cells. This example demonstrates that optimization of the formulation parameters is required in order to successfully lyophilize lipoplexes/polyplexes, which upon rehydration can be used for effective transfection of mammalian cells. One could envisage the design of optimum conditions for existing transfection reagents that will allow for transfection efficiency of multiple DNA together with ability to lyophilize the complex and act as a carrier for supplementary DNA.

### Example 2

### Carrier Lyophilized Powder (DTCPTA/DiPPE and 3 Plasmids) to which One Additional Free Plasmid (pORF9-hTNFa) is Added

The aim of this example was to evaluate the capacity of our lyophilized product (containing multiple DNA plasmids and a transfection reagent) to serve as a carrier for free additional DNA plasmid. The lyophilizate is rehydrated with a solution containing a DNA plasmid coding for a cytokine, as a reporter gene. The experiment compares the transfection efficiency of a) free additional plasmid as part of rehydrated carrier product with b) freshly prepared complex containing one DNA plasmid and a transfection reagent, c) a freshly prepared complex containing 4 DNA plasmids and a transfection reagent. The freshly prepared products serve as controls.

### Preparation of Transfection Mixture Using “Carrier” Product and Additional Free Plasmid

The “carrier” lyophilizate was prepared as described in example 1. In this example, an additional free plasmid was added and used to simultaneously introduce the complexed plasmids and additional free plasmid into animal cells. The lyophilizate “carrier” product was rehydrated with 1 ml of sterile water containing 15 μg of pORF9-hTNFa (3.917 kb; InvivoGen), a DNA plasmid coding for human tumor necrosis factor alpha. The DNA:lipid ratio is now 1:2 and the mixture has a negative charge. Final concentration of this mixture was 37.5 μg/ml total DNA.

### Freshly Prepared Transfection Mixtures

LyoVec™ transfection reagent was rehydrated with 2 ml of sterile water. Two different lipoplexes were prepared as follows;

1) 10 μg pORF9-hTNFa was added to 1 ml of rehydrated LyoVec™ transfection reagent (60 μg/ml) and left to incubate for 15 minutes at room temperature. Final concentration of this mixture is 10 μg/ml in pORF9-hTNFa DNA.

2) 2.5 μg pAcGFP1-N1, 2.5 μg pSEAP2-Control, and 2.5 μg pCMV-GLuc −2.5 μg pORF9-hTNFa was added to 1 ml of rehydrated LyoVec™ transfection reagent (60 μg/ml) and left to incubate for 15 minutes at room temperature. Final concentration of this mixture was 10 μg/ml total DNA.

### Transfection of 293T Cells

Transfection of 293T Cells (ATCC, cat no. CRL-11268) was carried out as follows: 300,000 cells/well were seeded in a 12-well plate, to which 100 μl of rehydrated carrier complex and free additional plasmid (3.75 μg total DNA; of which 1.5 μg is pORF9-hTNFa plasmid) was added directly into the culture medium drop wise and mixed by rocking the culture plate back and forth. For the freshly prepared transfection mixture, 100 μl of transfection mixture was added, containing 1 μg of total DNA per well as per instructed by manufacturer for optimal transfection efficiency. For the transfection mixture containing 4 plasmids, 0.25 μg is pORF9-hTNFa plasmid per well. For the transfection mixture containing 1 plasmid, 1 μg is pORF9-hTNFa plasmid per well. 24 hours after transfection, the culture media was replaced with fresh DMEM containing 10% heat inactivated FBS, in order to destroy alkaline phosphate present in serum which interferes with the quantification of SEAP. The cells were further incubated at 37° C. for a total of 48 hours to allow expression of the GFP, SEAP, and Gaussia luciferase transgenes.

Evaluation of reporter gene expression as described in example 1.

### Evaluation of TNF-Alpha Aerie Expression

Human α Immunoassay (R&D Systems; cat. no. DTA00C) was performed according to manufacturer's protocol. Briefly, this assay employs the quantitative ELISA technique, where a monoclonal antibody for TNFα has been pre-coated onto a microplate. Any TNFα present in the samples will bind to the immobilized antibody. Following wash steps, a secondary antibody and substrate were added. The absorbance readings were determined at 450 nm using a plate reader (FLUOStar OPTIMA).

TABLE 2
Showing % transfection efficiencies GFP, SEAP
and luciferase for lyophilized
product compared to a freshly prepared product. Mean ±
SD from three independent
transfection experiments.

| | | Freshly | Freshly |
|---|---|---|---|
| | | prepared | prepared |
| | Lyophilized | four plasmid | one plasmid |
| | product | product | product |
| GFP | 4,000 ± 500 | 4,500 ± 500 | Not |
| (fluorescence | | | applicable |
| Abs, 485 nm) | | | |
| SEAP(O.D. | 1 ± 0.2 | 1.2 ± 0.3 | Not |
| 650 nm) | | | applicable |
| Luciferase | 200,000 ± 600 | 350,000 ± 700 | Not |
| (RLU) | | | applicable |
| TNF (ng/ml) | 20 ± 8 | 30 ± 6 | 50 ± 10 |

### Stability Study

This experiment was performed on Hela, HEK293T and B16 cell line over a 6 month period to assess stability of the lyophilized and resuspended formulations. Comparable results were obtained demonstrating stability of this formulation.

### Conclusion

The results presented in table 2 indicate that the lyophilized carrier product allows expression of a reporter genes encoded on the complexed plasmids and the cytokine encoded on the additional free plasmids into 293T Cells with the same efficiency as the one obtained with the freshly prepared complex. These results are of interest to researchers working with cells of unknown or variable transfection efficiencies. The plasmids coding for the reporter genes as described in this example could constitute multiple internal controls allowing assessment of transfection efficiency by various mean like FACS, luminescence or colorimetric analysis.

### Example 3

### Application for Lentiviral Vector Production Using Lyophilized Carrier Powder (Transfection Reagent and 2 Plasmids) to which 1 Plasmid is Added

In this example, we aim to show that this formulation can be applied to the production of lentiviral particles. The lyophilized carrier product contains two plasmids containing packaging vector pCMV-8.93 (13.457 kb) and envelope vector pCMV-VSVG (6.363 kb)., for which the plasmid maps are in the public domain (www addgene.com) and a transfection reagent DTCPTA/DiPPE (components of LyoVec™ transfection reagent, InvivoGen). The carrier product was rehydrated with a plasmid coding for a reporter gene, GFP. The ability to produce lentiviral particles of the rehydrated carrier product was compared to that of a freshly prepared transfection solution (3 plasmids required for lentiviral vector production and LyoVec™/calcium phosphate transfection solution). The calcium phosphate method is taken as a reference transfection method for lentiviral vector production.

### Preparation of Lyophilized Carrier Product

The lyophilizate was prepared as follows; 12.5 μg pCMVdR8.2, 6.25 μg pCMV-VSVG was added to 500 μl of DTCPTA/DiPPE solution (125 μg/ml) in 0.45% (w/v) NaCl and 2.5% (w/v) saccharose). The mixture was placed at −80° C. for 24 hours prior to lyophilization. Lyophilization was carried out as follows, 20 hours at −30° C., 6 hours at −20° C., 8 hours at −10° C., and 6 hours at 35° C. The ratio of DNA:lipid is 1:3.3 (w/w) and has a negative charge.

### Transfection Using Carrier Lipoplex and Additional Free Plasmid

Following lyophilization, the lyophilizate was rehydrated using 12.5 μg of pLenti 6.2-GW/EmGFP control vector coding for GFP (7.883 kb; Invitrogen) in 1 ml of sterile water. The DNA:lipid ratio is 1:2 and lipoplex is negatively charged (final conc 31.25 μg/ml total DNA). The DNA-Lipid mixture was left to incubate for 15 minutes at room temperature. Transfection of 293T cells was carried out as follows: 8.10⁶ were seeded in a T75 flask, to which the 1 ml of the transfection mixture containing transfection reagent, complexed plasmids and additional free plasmid was added directly into the culture medium drop wise and mixed by rocking the culture plate back and forth. There was no need to change the culture media following transfection or need to wait for the cell to be adherent, as DTCPTA/DiPPE is not toxic to cells, unlike other transfection reagents. 36 hours later the cell supernatant was harvested.

Transfection with Calcium Phosphate

Transfection was carried out using the calcium phosphate co-precipitation method as described as a reference method of production as described by (Kutner et al., 2009). Briefly, 24 h prior to transfection 4.10⁶ 293 T cells were seeded in a T75 plate plate. The DNA/CaCl2 mixture was prepared (12.5 μg pCMV-8.2, 6.25 μg pCMV-VSVG, and 12.5 μg pLenti 6.2-GW/EmGFP with CaCl2) and added to Heppes Buffered Saline (HBS; Invitrogen, cat. no. 24020117) for a final volume of 1 ml. This mixture is added drop wise to 293T cells and left overnight at 37° C. Following the overnight incubation, the culture media was changed and following a further 24 hour incubation the cell supernatant was harvested.

### Freshly Prepared Transfection Mixture

LyoVec™ transfection reagent was rehydrated with 2 ml of sterile water. A lipoplex was freshly prepared as follows: 12.5 μg pCMV-8.2, 6.25 μg pCMV-VSVG, and 12.5 μg pLenti 6.2-GW/EmGFP was added to 1 ml of rehydrated LyoVec™ transfection reagent and left to incubate for 15 minutes at room temperature. The ratio of DNA:lipid is 1:6 (w/w) and is positively charged. Transfection of 293T cells was carried out as follows: 8.10⁶ were seeded in a T75 flask, to which the 1 ml of the transfection mixture containing transfection reagent, and plasmids was added directly into the culture medium drop wise and mixed by rocking the culture plate back and forth. There was no need to change the culture media following transfection or need to wait for the cell to be adherent, as DTCPTA/DiPPE is not toxic to cells, unlike other transfection reagents. 36 hours later the cell supernatant was harvested

### Transduction of HT1080

According to the method described by (Kutner et al., 2009), a human fibrosarcoma cell line, HT1080 cells (ATCC, cat. no. CCL-121) were transduced. Briefly, cells were plated at 5×10⁴ per well in 48-well plates, 24 hours prior to transduction. HT1080 were transduced for 2 h30 using serial dilution of the various lentiviral vector preparations. Cell were rinsed and left to incubate for a further 48 h prior to analysis.

### Flow Cytometric (FACS) Method to Titrate Lentiviral Vectors Expressing Fluorescent Proteins, Such as GFP

HT1080 cells were trypsinized. The cell suspension was analyzed by FACS analysis (FACS Canto™, BD BioSciences). The titer was calculated in transducing units (TU) per ml as described in (Kutner et al., 2009). Briefly, TU/ml=(F×N×D×1,000)/V, where F=percentage of fluorescent cells (GFP or DsRed), N=number of cells at the time of transduction, D=fold dilution of vector sample used for transduction, and V=volume (μl) of diluted sample added to each well for transduction.

Titration Assay Based on p24

Serial dilutions of the samples to be tested were prepared. Dilutions ranging from 10⁻³ to 10⁻⁶ for unconcentrated samples were prepared. The p24 levels in the diluted samples were determined using an INNOTEST® HIV Antigen mAB (INNOGENETICS) according to the protocol recommended by the manufacturer.

TABLE 3
Showing lentiviral particle titrations and p24
protein levels. Mean ± SD from three
independent transfection experiments.

| | Calcium | Fresh | Carrier |
|---|---|---|---|
| | phosphate | LyoVec ™ | LyoVec ™ |
| Titration (TU/ml) | 3 ± 2.1 × 10⁶ | 3.6 ± 1.6 × 10⁶ | 2.9 ± 1 × 10⁶ |
| P24 ng/ml | 10 ± 2 | 11 ± 3 | 9 ± 2 |

### Stability Study:

This experiment was performed several time over a 6 month period using the same batch of lyophilized product kept at 4 C. No reduction in titer was observed, demonstrating stability of the product.

### Conclusion

The above example proves that it is possible to use the lyophilized carrier product for lentiviral vector production. This formulation will have numerous advantages, such as ease-of-storage, stability, ease-of-use, reproducibility of a standardized formulation and good manufacturing practice (GMP) compliance. Results are similar for the lyophilized formulation and the reference production protocol using calcium phosphate.

### Example 4

### Application for Lentiviral Vector Production Using Lyophilized Carrier Powder (LyoVec™ and 3 Plasmids) to which One Additional Free Plasmid is Added

In this example, we extend the scope of the patent to a four plasmid formulation in the packaging mix instead of the previous three. The packaging plasmids in this example are commercially available from Invitrogen.

### Preparation of Carrier Product

The lyophilizate was prepared as follows; 8.5 μg LP1 (8.889 kb; Invitrogen), 6 μg pLP2 (4.180 kb; Invitrogen), and 8.5 μg pLP-VSVG (5.821 kb; Invitrogen) was added to 500 μl of DTCPTA/DiPPE solution (125 μg/ml) in 0.45% (w/v) NaCl and 2.5% (w/v) saccharose). The mixture was placed at −80° C. for 24 hours prior to lyophilization. Lyophilization was carried out as follows, 20 hours at −30° C., 6 hours at −20° C., 8 hours at −10° C., and 6 hours at 35° C. The ratio of DNA:lipid is 1:2.7 (w/w) and has a negative charge

### Transfection of 293F Cells Using Carrier Lipoplex and Additional Free Plasmid

Following lyophilization, the lyophilizate was rehydrated using 8 μg of the expression plasmid pTRIP-EF1α-hlFNb (Brule et al., 2007) in 1 ml of sterile water. The DNA:lipid ratio is 1:2 and lipoplex is negatively charged (final conc 31.25 μg/ml total DNA). The DNA-Lipid mixture was left to incubate for 15 minutes at room temperature. Transfection of 293T cells was carried out as follows: 8.10⁶ were seeded in a T75 flask, to which the 1 ml of the transfection mixture containing transfection reagent, complexed plasmids and additional free plasmid was added directly into the culture medium drop wise and mixed by rocking the culture plate back and forth. 36 hours later cell supernatant was harvested

### Transfection of 293T Cells Using FuGENE® HD Transfection Reagent

Transfection was carried out according to the protocol described in Biotechniques Protocol Guide 2009 (p. 31). Briefly, 4.10⁶ 293T cells were seeded at 50% confluence in a T75 plate. 10.5 μg pLP1, 7 μg pLP2, 10.5 μg pLP-VSVG, and 10 μg pTRIP-EF1α-hlFNb was mixed in 2 ml of serum free culture media (OptiMem) with 100 μl of FuGENE® HD transfection reagent. 850 μl of this mixture is added drop wise to 293T cells and left overnight at 37° C. Following the overnight incubation, the culture media was changed with fresh growth medium incubated a further 24 hour prior to supernatant harvest.

### Transduction of THP-1 Cells

According to the method described by (Kutner et al., 2009), THP-1 cells were transduced. Briefly, cells were seeded at 5×10⁴ per well in 48-well plates, 24 hours prior to transduction. THP-1 were transduced by adding 0.5, 5 and 50 μl aliquots, respectively, of the diluted vector suspension per well.

### Titration Assay Based on p24

Titration by p24 as described in example 4.

### Assessment of IFNβ Expression Using ELISA

48 hours following the transduction, the THP-1 cell supernatant was collected. IFNβ expression was assessed using human IFN-beta ELISA kit (R&D Systems, cat. no. 41410-1). Briefly, this kit quantitates human IFNβ in cell supernatant using an ELISA with streptavidin conjugated to horseradish peroxidase (HRP). The absorbance readings were determined at 450 nm using a plate reader (FLUOStar OPTIMA).

TABLE 4
Showing IFNβ and p24 protein levels. Mean ± SD from three independent
transfection experiments.

| | Carrier LipoGen ™ | FuGENE ® HD |
|---|---|---|
| IFNβ ng/ml | 15 ± 2 | 12 ± 3 |
| P24 ng/ml | 10 ± 2 | 11 ± 3 |

### Conclusion

ELISA for IFN showed that the lentiviral particles were functional, using equal amounts of particles based on the p24 titration. Results indicate that the ability to make lentiviral particles of the lyophilized “carrier” product with the additional free plasmid was similar to that of FuGENE® HD. This example indicates that it is possible to produce lentiviral particles using our lyophilized carrier product, using a cationic lipid transfection reagent and different numbers of the packaging plasmids.

### Example 5

### Application for Non-Integrating Lentiviral Vector Production Using Lyophilized “Carrier” Product Containing a Polymeric Transfection Reagent and 2 Plasmids to which an Additional Free Plasmid

In this example, we extend the scope of the patent to another transfection reagent, family by using cationic polymer (in vivo-jetPEI™, PolyPlus Transfection; cat. no. 201-10) instead of a lipid-based formulation. Example 5 also indicates that it is possible to produce non-integrating lentiviral vectors using a pseudotyping plasmid pCMV-VSV-G (6.363 kb) and a plasmid coding for the accessory proteins including a mutated integrase pCMV.8.93INTD6V (8551 kb; InvivoGen, not commercially available) to which an expression plasmid, pLenti 6.2-GW/EmGFP control vector (7.883 kb; Invitrogen, cat. no. V369020), is added as an additional free plasmid.

### Preparation of Lyophilizate Containing Transfection Reagent (PEI) and Plasmid DNA

The lyophilizate was prepared as follows; 6.25 μg pCMV-VSV-G and 12.5 μg pCMV.8.93INTD6V was added to 500 μl of a 6 mM in vivo-jetPEI™ solution in 5% (w/v) glucose. The mixture was placed at −80° C. for 24 hours prior to lyophilization. Lyophilization was carried out as follows, 20 hours at −30° C., 6 hours at −20° C., 8 hours at −10° C., and 6 hours at 35° C. The overall charge of the in vivo-jetPEI™/DNA complex is cationic.

### Transfection of 293T Cells Using Rehydrated Carrier Product

Following lyophilization, the lyophilizate was rehydrated using 10 μg of pLenti 6.2-GW/EmGFP control vector coding for GFP (7.883 kb; Invitrogen) in 1 ml of sterile water. The DNA-Lipid mixture was left to incubate for 15 minutes at room temperature. Transfection of 293T cells was carried out as follows: 8.10⁶ were seeded in a T75 flask, to which the 1 ml of the transfection mixture containing transfection reagent, complexed plasmids and additional free plasmid was added directly into the culture medium drop wise and mixed by rocking the culture plate back and forth and left overnight at 37° C. Following the overnight incubation, the culture media was changed and following a further 24 hour incubation the cell supernatant was harvested.

Transfection with Calcium Phosphate

Transfection was carried out using the calcium phosphate co-precipitation method as described as a reference method of production as described by (Kutner et al., 2009). Briefly, 24 h prior to transfection 4.10⁶ 293 T cells were seeded in a T75 plate plate. The DNA/CaCl2 mixture was prepared (12.5 μg pCMV.8.93INTD6V, 6.25 μg pCMV-VSVG, and 12.5 μg pLenti 6.2-GW/EmGFP with CaCl2) and added to Heppes Buffered Saline (HBS; Invitrogen, cat. no. 24020117) for a final volume of 1 ml. This mixture is added drop wise to 293T cells and left overnight at 37° C. Following the overnight incubation, the culture media was changed and following a further 24 hour incubation the cell supernatant was harvested.

### Transduction of HT1080

Transduction was performed as in example 3.

### Flow Cytometric (FACS) Method to Titrate Lentiviral Vectors Expressing Fluorescent Proteins, Such as GFP

Titration was performed as in example 3.

### Flow Cytometric (FACS) Method to Follow Expression of Fluorescent Proteins, Such as GFP by Transduced Cells in Culture

HT1080 cells were seeded at 5.10⁴ cells per well in a 48 wells plate and transduced with integrative lentiviral vector or non-integrative lentiviral vector at MOI 10. After 12 hours of transduction, cells were washed twice and maintained in culture for 30 days including passages when cells were confluent. EGFP expression in transduced cells was analyzed at different times of culture by flow cytometry.

### Results & Conclusion

TABLE 5
Showing lentiviral particle titrations. Mean ± SD from three
independent transfection experiments.

| | Calcium phosphate | Fresh PEI ™ | Carrier PEI ™ |
|---|---|---|---|
| Titration (TU/ml) | 1.7 ± 0.8 × 10⁵ | 0.8 ± 0.4 × 10⁵ | 0.9 ± 0.5 × 10⁵ |

### Results & Conclusion

This example indicates that it is possible to use a cationic polymer, such as linear PEI, in the carrier product. Furthermore, this example demonstrates that it is possible to produce modified viral vectors, such as non-replicating lentiviral vectors using the rehydrated carrier product.

### REFERENCES

Throughout this application, various references describe the state of the art to which this invention pertains. The disclosures of these references are hereby incorporated by reference into the present disclosure.

### US Patent Documents

U.S. Pat. No. 5,994,136 11/1999 Naldini et al.
U.S. Pat. No. 6,013,516 01/2000 Verma et al.
U.S. Pat. No. 6,165,782 12/2000 Naldini et al.
U.S. Pat. No. 6,428,953 08/2002 Naldini et al.
U.S. Pat. No. 6,790,657 09/2004 Arya
U.S. Pat. No. 6,726,926 04/2004 Yajima et al.
U.S. Pat. No. 6,924,144 08/2005 Naldini et al.
U.S. Pat. No. 7,083,981 08/2006 Naldini et al.
U.S. Pat. No. 7,220,578 05/2007 Kafri & Ma
U.S. Pat. No. 7,226,780 06/2007 Arya
U.S. Pat. No. 7,250,299 07/2007 Naldini et al.
U.S. Pat. No. 7,276,359 10/2007 Musunuri et al.

### Relevant Patent Applications

US 2003/0092652 A1 Protected one-vial formulation for nucleic acid molecules, methods of making the same by in-line mixing, and related products and methods.
US 2005/0002998 A1 Method for improving stability and shelf-life of liposome complexes.
US 2008/0089863 A1 Non-integrative and non-replicative lentivirus, preparation and use thereof.

### Other Publications

Allison S. D., &. Anchordoquy T. J., J Pharm Sci. 89:682-691 (2000).
Anchordoquy T. J., et al., Arch Biochem Biophys. 348:199-206 (1997).
Behr J. P. Bioconjug Chem. 5: 382-389 (1994).
Biotechniques Protocol Guide 2009 (p. 13).
Birchall J. C., et al., Int J Pharm 183, 195-207 (1999).
Brule, F., et al., Biochem Pharmacol 74, 898-910 (2007).
Burns J. C. et al., Proc Natl Acad Sci USA. 90: 8033-8037 (1993).
Bushman F. Science 267, 1443-1444 (1995).
Bushman F. D., & Miller, M. D. J Virol 71, 458-464 (1997).
Cherng J. Y., et al., Int J Pharm, 183:25-8 (1999).
Cotton M., et al., Curr Opin Biotechnol. 4:705-710 (1993).
Dull T., et al., J Virol 72, 8463-8471 (1998).
Ewert K., et al., Adv Genet 53PA, 119-155 (2005).
Felgner J. H., et al., J Biol Chem. 269:2550-2561 (1994).
Felgner J. H., et al., Hum Gene Ther. 8:511-512 (1997).
Intra J. & Salem A. K. J Control Release 130:129-138 (2008).
Katz R. A., et al., Virology 217, 178-190 (1996).
Kopatz I., et al., J Gene Med 6, 769-776 (2004).
Kutcher R. H., et al., Nat Protoc 4, 495-505 (2009).
Li B. et al., J Pham Sci., 89: 355-364 (2000).
Lu R. & Nakajimaet al., J. Virol. 78, 658-668 (2004).
Masotti A., et al., Colloids Surf B Biointerfaces 68, 136-144 (2009).
Naldini L. et al., Proc Natl Acad Sci USA, 93:11382-8 (1996).
Nightingale S. J., et al, Mol Ther 13, 1121-1132 (2006).
Russell D. W. & Miller A. D. J. Virol. 70: 217-222 (1996).
Saenz D. T., et al., J Virol 78, 2906-2920 (2004).
Turek J., et al., J Gene Med 2, 32-40 (2000).
Vargas J., et al., Hum Gene Ther 15, 361-372 (2004).
Verma I. M., & Somia, N. Gene. Nature 389, 239-242 (1997).
Yanez-Munoz et al., Nat Med 12, 348-353 (2006).
Zennou V., et al., Cell 101:173-185 (2000).
Zufferey R., et al., Nat Biotechnol. 15:871-875 (1997).
Zufferey R., et al., J Virol 73:2886-2892 (1999).

What is claimed is:

1. A method for transfecting an animal cell, comprising the steps of:
a) rehydrating a lyophilized composition comprising a lyoprotectant and one or more plasmids complexed with a DNA transfection reagent;
b) adding to the rehydrated composition obtained in step a) one or more free plasmids; and
c) transfecting an animal cell with the mixture obtained in step b).

2. The method of claim 1, wherein the transfection reagent comprises a cationic polymer or a cationic lipid with or without a neutral helper lipid.

3. The method of claim 2, wherein the transfection reagent comprises a cationic lipid with a neutral helper lipid.

4. The method of claim 2, wherein the transfection reagent comprises a cationic polymer.

5. The method of claim 1, wherein the lyoprotectant is a carbohydrate.

6. The method of claim 1, wherein the lyophilized composition further comprises a salt.

7. The method of claim 1, wherein said lyophilized composition comprises two or more plasmids complexed with a DNA transfection reagent:
a packaging plasmid coding for a pseudotyping gene, and
one or more plasmids coding for one or more lentiviral accessory genes selected from the group consisting of gag, tat, pol and rev.

8. The method of claim 1, wherein lyophilized composition comprises three plasmids complexed with a DNA transfection reagent:
a plasmid coding for one or more lentiviral accessory genes selected from the group consisting of gag, tat and pol;
a plasmid coding for rev, and
a packaging plasmid coding for a pseudotyping gene.

9. The method of claim 1, wherein the one or more additional free plasmids added in step b) encode a protein or a RNA of interest.

10. A kit comprising:
a) a lyophilized composition comprising a lyoprotectant and one or more plasmids complexed with a DNA transfection reagent, and
b) an expression vector comprising a cloning site which enables the introduction of a nucleotide of interest.
